(19) United States
(12) Patent Application Publication
(10) Pub. No.: US 2026/0334919 A1
(43) Pub. Date: Oct. 29, 2026

(54) VESSEL AND LUMEN BORDER VIEWING ICON FOR INTRAVASCULAR IMAGING SYSTEM

(71) Applicant: Boston Scientific Scimed, Inc., Maple Grove, MN (US)

(72) Inventors: Brandon Matthew Zingsheim, Circle Pines, MN (US); Viola Kim Holman Narveson, Crystal, MN (US)

(73) Assignee: Boston Scientific Scimed, Inc., Maple Grove, MN (US)

(21) Appl. No.: 19/657,713

(22) Filed: Apr. 24, 2026

Related U.S. Application Data
(60) Provisional application No. 63/795,005, filed on Apr. 25, 2025.

(51) Int. Cl. A61B 8/00; A61B 8/08; A61B 8/12; G06T 7/13

(52) U.S. Cl. CPC A61B 8/465; A61B 8/0891; A61B 8/12; A61B 8/467; A61B 8/523; A61B 8/5246; G06T 7/13; G06T 2200/24; G06T 2207/10016; G06T 2207/10132; G06T 2207/30101

(57) ABSTRACT

A computer-implemented method and system for intravascular image navigation includes receiving intravascular image frames captured along a cardiac artery and receiving vessel and lumen border indications for the frames. The system generates a graphical user interface (GUI) displaying multiple graphical components including visualizations of first and second vessel borders and first and second lumen borders within cross-sectional views. A viewing button in the GUI enables transitioning between different display states - an enabled state showing all border visualizations and disabled states selectively hiding different combinations of the border visualizations. The viewing button allows quick toggling between the different visualization modes to enhance workflow efficiency and clinical decision-making flexibility.

### CROSS-REFERENCE TO RELATED APPLICATIONS

This application claims the benefit of priority under 35 U.S.C. §119 of U.S. Provisional Application No. 63/795,005, filed Apr. 25, 2025, the entire disclosure of which is hereby incorporated by reference.

### TECHNICAL FIELD

The present disclosure generally relates to intravascular imaging (IVI) systems, such as intravascular ultrasound (IVUS) imaging systems. Particularly, but not exclusive, the present disclosure relates to an improved graphical user interface for IVI systems.

### BACKGROUND

Intravascular ultrasound devices insertable into patients have proven diagnostic capabilities for a variety of diseases and disorders. For example, intravascular ultrasound (IVUS) imaging systems have been used as an imaging modality for diagnosing blocked blood vessels and providing information to aid medical practitioners in selecting and placing stents and other devices to restore or increase blood flow.

In general, IVUS imaging systems include a control module (with a pulse generator, an image acquisition and processing components, and a monitor), a catheter, and a transducer disposed in the catheter. The transducer-containing catheter is positioned in a lumen or cavity within, or in proximity to, a region to be imaged, such as a blood vessel wall or patient tissue in proximity to a blood vessel wall. The pulse generator in the control module generates electrical pulses that are delivered to the transducer and transformed to acoustic pulses that are transmitted through patient tissue. The patient tissue (or other structure) reflects the acoustic pulses and reflected pulses are absorbed by the transducer and transformed to electric pulses. The transformed electric pulses are delivered to the image acquisition and processing components and converted into images displayable on the monitor.

IVUS systems can be used to visualize segments of a vascular system and provide complementary views to extravascular imaging modalities, such as angiography, due to, for example, movement (e.g., a beating heart) or obstruction by one or more structures (e.g., one or more blood vessels not desired to be imaged).

Often IVI modalities (e.g., IVUS, optical coherence tomography, or the like) are used to visualize borders of the vessel of interest. Thus, there is a need for user interfaces, and particularly graphical user interfaces, that can quickly switch between views of the differing intravascular assessments (e.g., borders, stents, lesions, etc.)

### BRIEF SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

An intravascular image system may generate a graphical user interface (GUI) displaying multiple graphical components including visualizations of vessel borders and lumen borders within cross-sectional views. The system may also include a viewing button to quickly toggle between different display states.

In some embodiments, a computer-implemented method for the intravascular image navigation system has a processor that receives a series of intravascular image (IVI) frames, receives an indication of a border of a vessel and a border of a lumen for at least a first IVI frame, generates graphical indications including a cross-section view, vessel border visualization, lumen border visualization, and a viewing icon with multiple states, renders a GUI, and sends information to a display, where the GUI shows different visualizations based on the viewing icon state.

In further embodiments, the processor can receive an indication of activation of the viewing icon and send a subsequent information element comprising an indication of a state of the viewing icon, where activation changes the viewing icon state.

In further embodiments, the processor can receive indications of manual borders of the vessel and lumen from an input device, where these manual borders are shown when the viewing icon is in the second state but not shown when in the third state.

In further embodiments, when the viewing icon is in the first state, the GUI does not show the manual borders of the vessel or lumen.

In further embodiments, when the viewing icon is in a fourth state, the GUI does not show any of the vessel border, lumen border, manual vessel border, or manual lumen border visualizations.

In further embodiments, the processor can receive indications of one or more assessments for the first IVI frame, where these assessments are shown when the viewing icon is in the first state.

In further embodiments, when the viewing icon is in the fourth state, the GUI does not show the visualization of the assessments.

In further embodiments, the processor can detect the vessel border and lumen border based on the first IVI frame.

In further embodiments, the detection of vessel and lumen borders can be done by applying image processing techniques to the first IVI frame.

In further embodiments, the detection of vessel and lumen borders can be done using a machine learning model.

In some embodiments, an intravascular imaging system has a processor and memory with instructions that when executed cause the system to receive IVI frames, receive vessel and lumen border indications, generate a cross-section view, generate graphical indications including visualizations and a viewing icon with multiple states, render a GUI, and send display information, where the GUI shows different visualizations based on the viewing icon state.

In further embodiments, the system can receive viewing icon activation and send subsequent information about the icon state, where activation changes the state.

In further embodiments, the system can receive manual border indications for the vessel and lumen, where these are shown in the second state but not in the third state.

In further embodiments, when the viewing icon is in the first state, the GUI does not show the manual borders.

In some embodiments, an intravascular imaging system has a processor and memory with instructions that when executed cause the system to receive IVI frames captured along a cardiac artery, receive vessel and lumen border indications, generate views and graphical indications including a viewing icon with multiple states, render a GUI, and send display information, where the GUI shows different visualizations based on the viewing icon state.

In further embodiments, the system can receive viewing icon activation and send subsequent information about the icon state.

In further embodiments, the system can receive manual border indications that are shown in the second state but not in the third state.

In further embodiments, when the viewing icon is in the first state, the GUI does not show the manual borders.

In further embodiments, when the viewing icon is in a fourth state, the GUI does not show any border visualizations.

In further embodiments, when the viewing icon is in the first state, the GUI shows one or more assessments.

In further embodiments, when the viewing icon is in the fourth state, the GUI does not show the assessments.

In further embodiments, the processor can detect borders using image processing techniques.

In further embodiments, the processor can detect borders using a machine learning model.

In some embodiments, a method for an intravascular image navigation system has a processor that receives IVI frames captured along a cardiac artery, receives vessel and lumen border indications, generates graphical indications including a viewing icon with multiple states, renders a GUI, and sends display information, where the GUI shows different visualizations based on the viewing icon state.

In further embodiments, the processor can receive viewing icon activation and send subsequent state information.

In further embodiments, the processor can receive manual border indications that are shown in the second state but not in the third state.

In further embodiments, when the viewing icon is in the first state, the GUI does not show the manual borders.

In further embodiments, when the viewing icon is in a fourth state, the GUI does not show any border visualizations.

In further embodiments, the processor can receive indications of assessments that are included in the graphical indications.

In further embodiments, when the viewing icon is in the first state, the GUI shows the assessments.

In further embodiments, when the viewing icon is in the fourth state, the GUI does not show the assessments.

In further embodiments, the processor can detect the vessel and lumen borders based on the first IVI frame.

In further embodiments, the border detection can be done using image processing techniques.

In further embodiments, the border detection can be done using a machine learning model.

Some embodiments can be implemented as a computer-readable storage device comprising instruction, which when executed by a processor of an intravascular imaging system to implement the method of any of the embodiments described herein.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1A illustrates an example cardiac vasculature structure containing an IVUS catheter inserted into the one of the cardiac arteries.

FIG. 1B illustrates an IVUS imaging system including the IVUS catheter shown in FIG. 1A.

FIG. 2A illustrates an example series of IVUS image frames.

FIG. 2B illustrates a longitudinal representation of the example series of IVUS image frames of FIG. 2A.

FIG. 3 illustrates an IVUS navigation system.

FIG. 4 illustrates a method to depict graphical indications via a graphical user interface (GUI).

FIG. 5A illustrates a first example graphical user interface (GUI).

FIG. 5B illustrates a second example GUI.

FIGS. 6A, 6B, and 6C illustrate an example viewing icon having multiple states.

FIGS. 7A, 7B, 7C, and 7D illustrate another example viewing icon having multiple states.

FIG. 8 illustrates a computer-readable storage medium.

FIG. 9 illustrates a diagrammatic representation of a machine.

### DETAILED DESCRIPTION

The foregoing has broadly outlined the features and technical advantages of the present disclosure such that the following detailed description of the disclosure may be better understood. It is to be appreciated by those skilled in the art that the embodiments disclosed may be readily utilized as a basis for modifying or designing other structures for carrying out the same purposes of the present disclosure. The novel features of the disclosure, both as to its organization and operation, together with further objects and advantages will be better understood from the following description when considered in connection with the accompanying figures. It is to be expressly understood, however, that each of the figures is provided for the purpose of illustration and description and is not intended as a definition of the limits of the present disclosure.

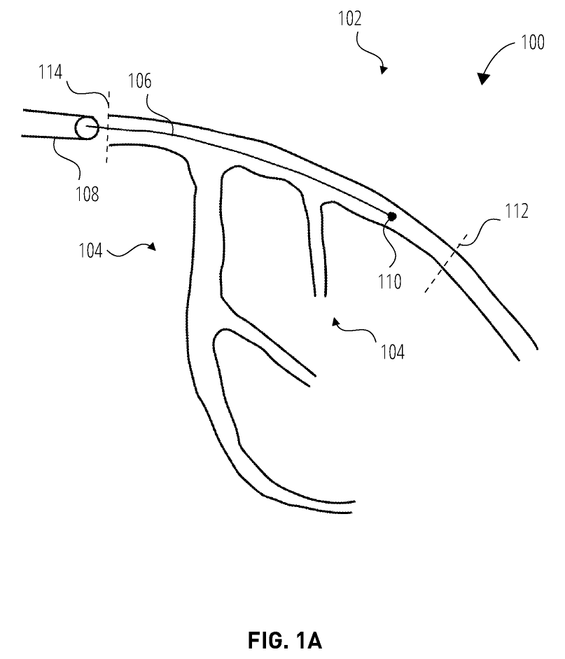

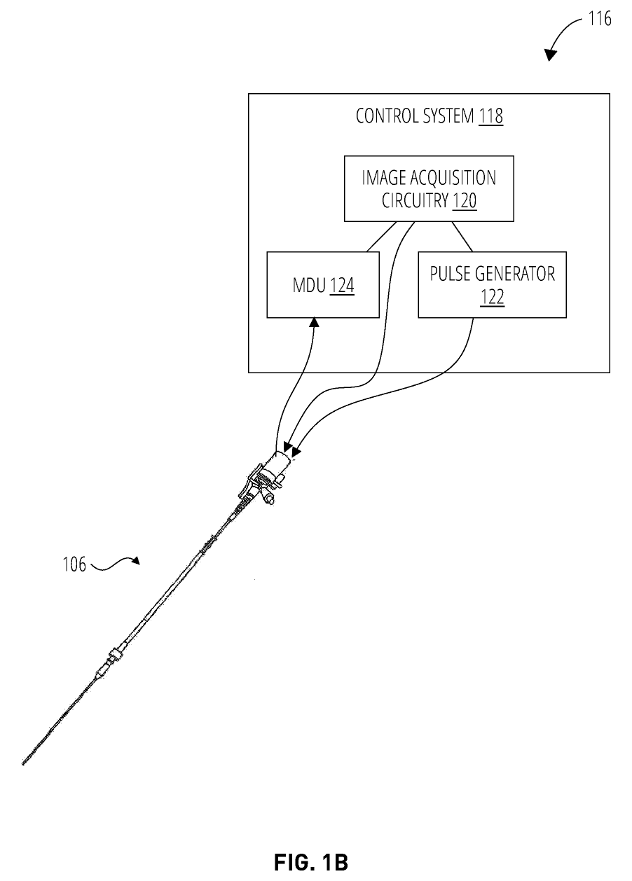

FIG. 1A illustrates a cardiac vasculature structure 100 having a cardiac artery 102 (e.g., cardiac artery, vessel, or the like) with several side branches 104 (e.g., descending arteries, marginal arteries, vessels, or the like) branching off the cardiac artery 102.

An intravascular imaging catheter (e.g., IVUS imaging catheter 106) is inserted into the cardiac artery 102 via a guide catheter 108 and images of the cardiac artery 102 are captured. The IVUS imaging catheter 106 includes an ultrasound transducer 110 disposed on a distal end of the IVUS imaging catheter 106. The IVUS imaging catheter 106 can be pulled back from a distal point 112 in the cardiac artery 102 to a proximal point 114 in the cardiac artery 102 and intravascular image frames are captured while the IVUS imaging catheter 106 is pulled back.

The present disclosure can be implemented for any intravascular imaging modality. However, the balance of the disclosure uses IVUS imaging (e.g., captured via IVUS imaging catheter 108, or the like) as an exemplary modality. This is done for ease of discussion only. To aid this discussion, FIG. 1B illustrates an IVUS imaging system 116. The IVUS imaging system 116 is depicted including IVUS imaging catheter 106, which can be coupled to a control system 118. The control system 118 may include, for example, an image acquisition circuitry 120, a pulse generator 122, and a motor drive unit (MDU) 124. In at least some embodiments, the pulse generator 122 forms electric pulses that may be input to one or more transducers (e.g., ultrasound transducer 110, or the like) disposed on IVUS imaging catheter 106.

With some embodiments, ultrasound transducer 110 can be part of an imaging core and coupled to the proximal end of IVUS imaging catheter 106 via a drive cable or drive shaft. Mechanical energy from the motor drive unit 124 can be used to rotate the imaging core (and thus the ultrasound transducer 110).

The electrical pulses generated by pulse generator 122 can be delivered to the ultrasound transducer 110 while the ultrasound transducer 110 is rotated by the MDU 124. The electrical signals are transformed by the ultrasound transducer 110 into acoustic pulses that are transmitted through the tissue of cardiac vasculature structure 100. The tissue of cardiac vasculature structure 100 reflects the acoustic pulses, which are absorbed by the ultrasound transducer 110 and transformed into electric pulses. The transformed electric pulses are delivered to the image acquisition circuitry 120 and converted into IVUS images displayable on a monitor.

For example, image acquisition circuitry 120 can be configured to map scan line samples (e.g., radial scan line samples, or the like) to a two-dimensional Cartesian grid, which can be used as the basis for a series of IVUS images that can be displayed for a user. In at least some embodiments, the image acquisition circuitry 120 may also be used to control the functioning of one or more of the other components of the control system 118. For example, the image acquisition circuitry 120 may be used to control at least one of the frequency or duration of the electrical pulses transmitted from the pulse generator 122, and the rotation rate of the imaging core by the MDU 124. Additionally, where the IVUS imaging system 116 is configured for automatic pullback, the MDU 124 can control the velocity and/or length of the pullback region (e.g., distal point 112 to proximal point 114, or the like).

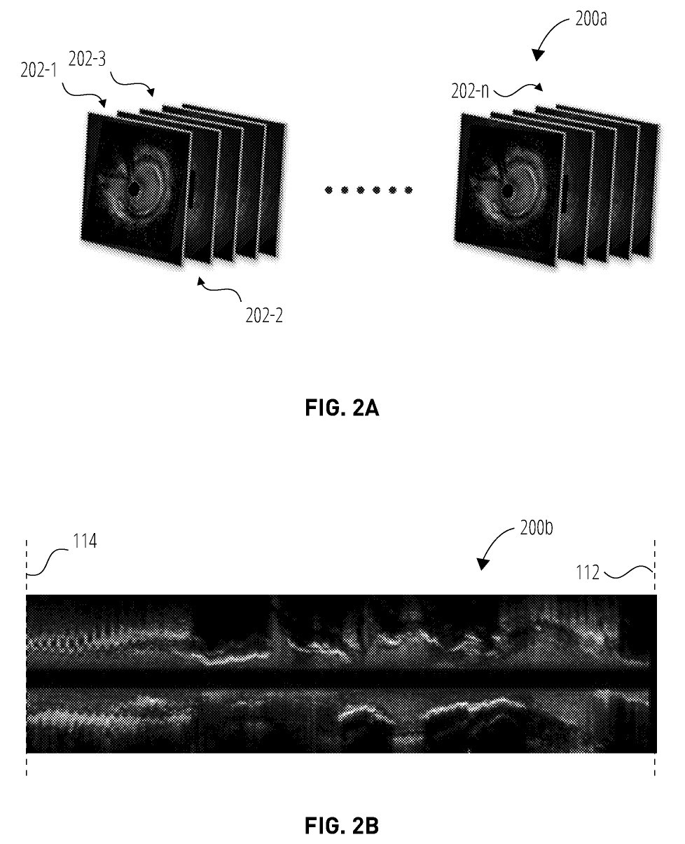

FIGS. 2A and 2B illustrate a series of IVUS image frames 200a and a longitudinal view 200b of the series of IVUS image frames 200a. As described above, several IVUS image frames can be captured while the IVUS imaging catheter 106 is moved through the cardiac artery 102. For example, FIG. 2A depicts the series of IVUS image frames 200a including IVUS image frames 202-1, 202-2, 202-3 to 202-n, where n is a positive integer, in this case, greater than ten (10). In practice, n can be any positive integer greater than or equal to two (2) but will often be greater than one hundred.

The series of IVUS image frames 200a can be stacked or grouped and depicted longitudinally to represent a longitudinal slice of the cardiac artery 102 from the distal point 112 to the proximal point 114. For example, FIG. 2B illustrates longitudinal view 200b which shows a longitudinal slice of cardiac artery 102 from distal point 112 to proximal point 114 formed from the series of IVUS image frames 200a.

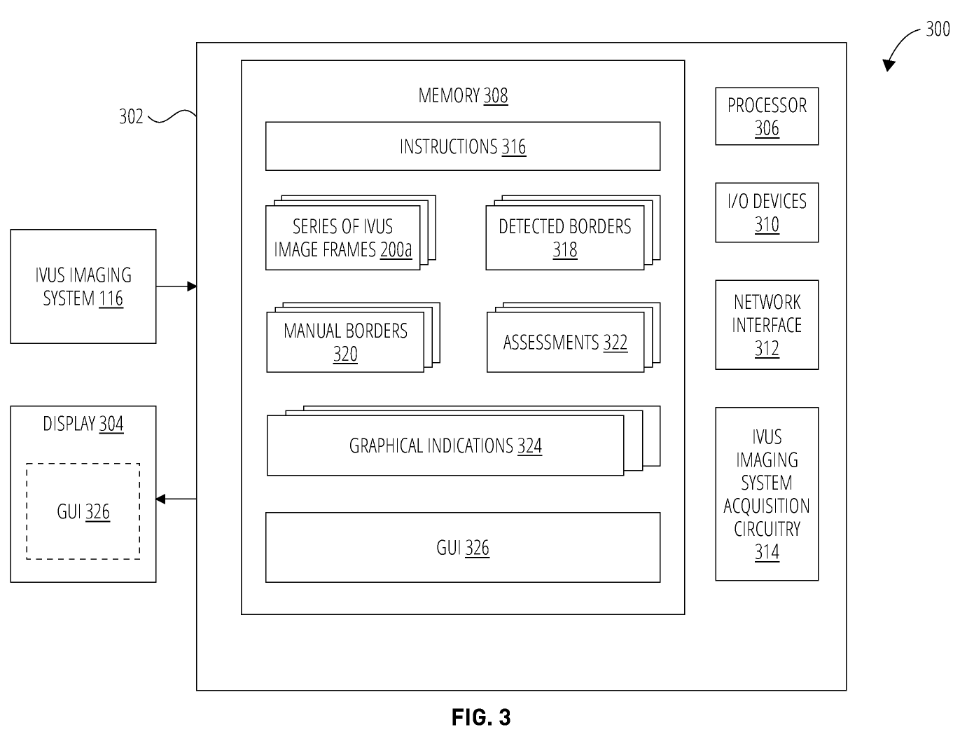

FIG. 3 illustrates an IVUS navigation system 300, which can be implemented to provide a GUI to display and allow interrogation of a vessel border and/or lumen border. In general, the IVUS navigation system 300 is a system for processing, annotating, and/or presenting IVUS images. IVUS navigation system 300 can be implemented in a commercial IVUS guidance or navigation system, such as, for example, the AVVIGO^{TM} Guidance System available from Boston Scientific®. Among other reasons, the present disclosure provides advantages over prior or conventional IVUS navigation systems in that a viewing button (also referred to as a viewing icon) is configured to quickly toggle between different visualization modes, or states. The inclusion of the viewing icon may enhance and improve user interface functionality and user control over displayed information, leading to a more efficient clinical workflow, improvements in clinical decision-making, and flexible viewing options for various clinical scenarios.

IVUS navigation system 300 includes a computing device 302 and display 304. Optionally, IVUS navigation system 300 includes an IVUS imaging system, such as IVUS imaging system 116. Where IVUS navigation system 300 includes IVUS imaging system 116, IVUS navigation system 300 could be implemented as part of control system 118 or alternatively, control system 118 could be implemented as part of computing device 302 of IVUS navigation system 300. IVUS navigation system 300 will be described with reference to cardiac vasculature structure 100 and IVUS imaging system 116 of FIGS. 1A and 1B as well as series of IVUS image frames 200a and longitudinal view 200b of FIGS. 2A and 2B for clarity and ease of discussion. However, it is noted that IVUS navigation system 300 could be implemented for use with another IVUS imaging system or even another intravascular imaging system utilizing a different imaging modality than IVUS imaging system 116.

The computing device 302 can be any of a variety of computing devices. In some embodiments, as noted above, computing device 30 can be incorporated into and/or implemented by a console to be coupled to an intravascular imaging device (e.g., IVUS imaging system 116, IVUS imaging catheter 106, or the like). With some embodiments, computing device 302 can be a workstation or server communicatively coupled to IVUS imaging system 116. With still other embodiments, computing device 302 can be provided by a cloud-based computing device, such as, by a computing as a service system accessibly over a network (e.g., the Internet, an intranet, a wide area network, or the like). Computing device 302 can include processor 306, memory 308, input and/or output (I/O) devices 310, network interface 312, and IVUS imaging system acquisition circuitry 314.

The display 304 may include any of a variety of devices arranged to display graphical information, such as, a light emitting diode (LED) display, or the like). It is to be appreciated that although display 304 is depicted separate from computing device 302, display 304 could be implemented as part of computing device 302 or be distinct from computing device 302.

The processor 306 may include circuity or processor logic, such as, for example, any of a variety of commercial processors. In some examples, processor 306 may include multiple processors, a multi-threaded processor, a multi-core processor (whether the multiple cores coexist on the same or separate dies), and/or a multi-processor architecture of some other variety by which multiple physically separate processors are in some way linked. Additionally, in some examples, the processor 306 may include graphics processing portions and may include dedicated memory, multiple-threaded processing and/or some other parallel processing capability. In some examples, the processor 306 may be an application specific integrated circuit (ASIC) or a field programmable integrated circuit (FPGA).

The memory 308 may include logic, a portion of which includes arrays of integrated circuits, forming non-volatile memory to persistently store data or a combination of non-volatile memory and volatile memory. It is to be appreciated that the memory 308 may be based on any of a variety of technologies. In particular, the arrays of integrated circuits included in memory 308 may be arranged to form one or more types of memory, such as, for example, dynamic random-access memory (DRAM), NAND memory, NOR memory, or the like.

The I/O devices 310 can be any of a variety of devices to receive input and/or provide output. For example, I/O devices 310 can include, a keyboard, a mouse, a joystick, a foot pedal, a display, a touch enabled display, a haptic feedback device, an LED, or the like.

The network interface 312 can include logic and/or features to support a communication interface. For example, network interface 312 may include one or more interfaces that operate according to various communication protocols or standards to communicate over direct or network communication links. Direct communications may occur via use of communication protocols or standards described in one or more industry standards (including progenies and variants). For example, network interface 312 may facilitate communication over a bus, such as, for example, peripheral component interconnect express (PCIe), non-volatile memory express (NVMe), universal serial bus (USB), system management bus (SMBus), SAS (e.g., serial attached small computer system interface (SCSI)) interfaces, serial AT attachment (SATA) interfaces, or the like. Additionally, network interface 312 can include logic and/or features to enable communication over a variety of wired or wireless network standards. For example, network interface 312 may be arranged to support wired communication protocols or standards, such as, Ethernet, or the like. As another example, network interface 312 may be arranged to support wireless communication protocols or standards, such as, for example, Wi-Fi, Bluetooth, 5G, or the like.

The IVUS imaging system acquisition circuitry 314 may include circuity including custom manufactured or specially programmed circuitry configured to receive or receive and send signals with IVUS imaging system 116, including indications of intravascular images, intravascular image frames, or a series of intravascular image frames. For example, IVUS imaging system acquisition circuitry 314 can include image acquisition circuitry 120 and/or pulse generator 122.

Memory 308 can include instructions 316, series of IVUS image frames 200a, detected borders 318, manual borders 320, assessments 322, graphical indications 324, and graphical user interface (GUI) 326.

During operation, processor 306 can execute instructions 316 to cause computing device 302 to receive (e.g., from IVUS imaging system 116, or the like) a recording of an “IVUS run” and store the recording as the series of IVUS image frames 200a in memory 308. For example, processor 306 can execute instructions 316 to receive information elements from IVUS imaging system 116 comprising indications of IVUS image frames 202-1, 202-2, 202-3 to 202-n, captured by IVUS imaging catheter 106 while IVUS imaging catheter 106 is pulled through cardiac artery 102 from the distal point 112 of the cardiac artery 102 to the proximal point 114 of the cardiac artery 102. It is to be appreciated that the series of IVUS image frames 200a can be stored in a variety of image formats or even non-image formats or data structures.

The present disclosure provides to process the series of IVUS image frames 200a to detect a detected vessel border and/or a detected lumen border (collectively “detected borders”) 318 via a GUI. Processor 306 can execute instructions 316 to generate border graphical indications 324 for the detected borders 318 and/or generate GUI 326 from the border graphical indications 324. In general, the border graphical indications 324 and/or GUI 326 can comprise visual indications of the series of IVUS image frames 200a, the detected borders 318, and assessments 322 (described further below) for the detected borders 318. In other embodiments, processor 306 can execute instructions 316 to detect borders represented in the series of IVUS image frames 200a and store the detected borders as detected borders 318.

With some embodiments, processor 306 can execute instructions 316 to receive indications of the vessel border and/or detected lumen border detected from the series of IVUS image frames 200a and store the indications as detected borders 318. In other embodiments, processor 306 can execute instructions 316 to detect borders represented in the series of IVUS image frames 200a and store the detected borders as detected borders 318.

In some embodiments, processor 306 can execute instructions 316 to identify the borders of the vessel and/or lumen represented in the IVUS image frames 200a and store the detected borders as detected borders 318. In some embodiments, processor 306 can execute instructions 316 to detect the borders using a machine learning (ML) models trained to identify vessel and/or lumen borders from IVUS images. As another example, processor 306 can execute instructions 316 to detect the borders from the series of IVUS image frames 200a using image processing algorithms that identify vessel features in frames of a series of frames. With yet another example, processor 306 can execute instructions 316 to identify the detected borders 318 from the series of IVUS image frames 200a using a combination and image processing techniques and ML models.

Further, the present disclosure contemplates that a user of the IVUS navigation system 300 may provide (or draw) borders of the vessel and/or lumen represented in the series of IVUS image frames 200a. For example, the processor 306 can execute instructions 316 to receive indications of manual borders 320 (e.g., via I/O devices 310, or the like).

With some embodiments, processor 306 can execute instructions 316 to detect borders for each frame of the series of IVUS image frames 200a while receiving indications of manual borders for only select frames of the series of IVUS image frames 200a.

Additionally, the disclosure provides to generate (e.g., automatically, or via received input) various assessments 322 related to the vessel and/or lumen represented in the series of IVUS image frames 200a. The assessments 322 can include, for example, raw vessel and/or lumen areas, smoothed vessel and/or lumen areas, a plaque burden, or the like. With some embodiments, processor 306 can execute instructions 316 to determine the assessments 322 as part of detecting the borders of the vessel and/or lumen.

Detection of borders 318 and derivation of assessments 322 is beyond the scope of this disclosure. However, an example of processing IVUS images to detect borders and derive assessments are described in more detail in United States Patent Application Publication No. 2023/0112017, titled “Medical Device Systems for Automatic Lesion Assessment” and filed on Oct. 5, 2022, which is incorporated herein by reference in their entirety.

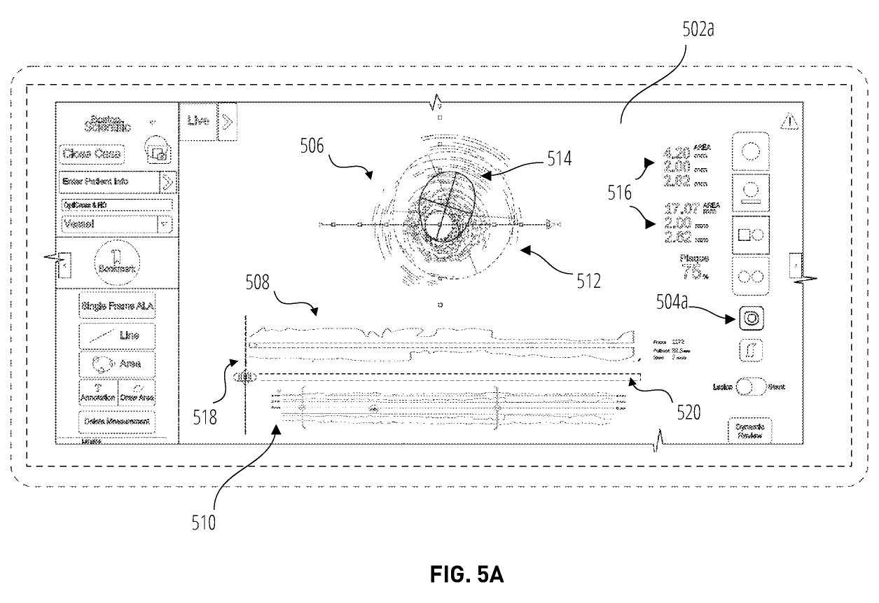

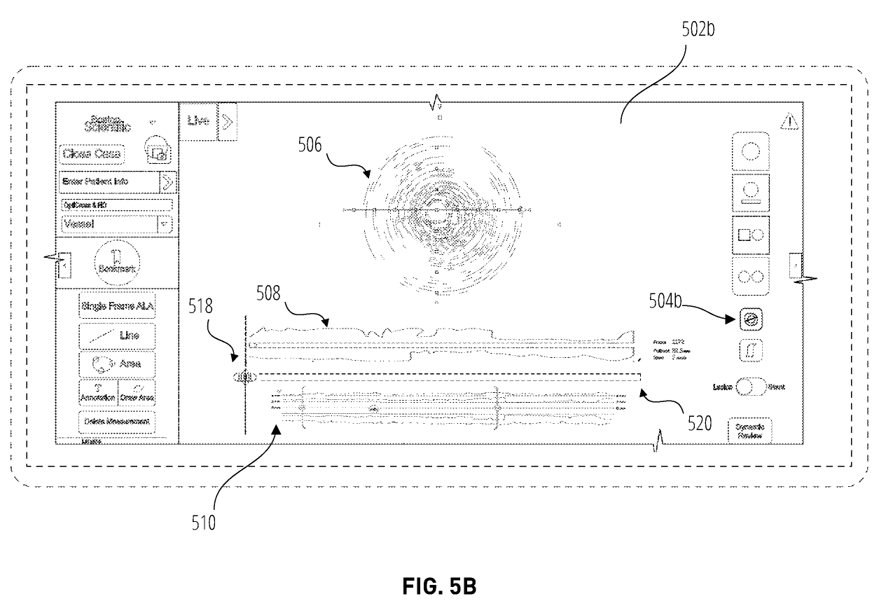

The present disclosure further provides to display visual indications of the detected borders 318, manual borders 320, and/or assessments 322 via a GUI. To that end, processor 306 can execute instructions 316 to generate graphical indications 324 and to render GUI 326 from the graphical indications 324. In general, the graphical indications 324 can comprise visual indications of a frames or frames of the series of IVUS image frames 200a, detected borders 318, manual borders 320, and assessments 322. Further, the graphical indications 324 can include a visualization of a viewing icon (see FIGS. 5A and 5B to 8A and 8B) having a number of states. The states of the viewing icon can correspond to various ones of the graphical indications 324 to be displayed on the GUI 326.

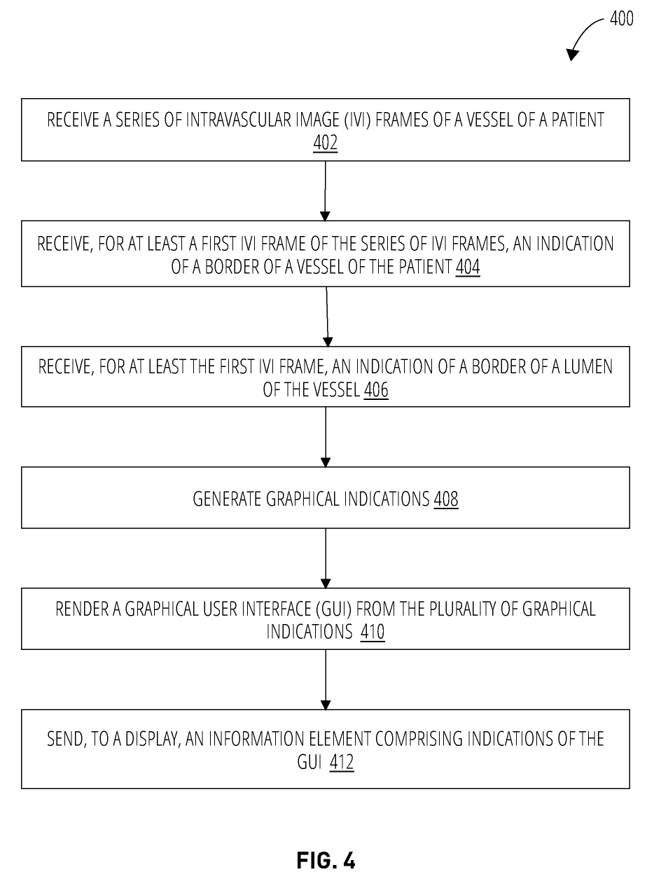

FIG. 4 illustrates a method or logic flow 400 to display visual indications of borders and IVUS image frames in a GUI. The logic flow 400 can be implemented by IVUS navigation system 300 and will be described with reference to IVUS navigation system 300 for clarity of presentation. However, it is noted that logic flow 400 could also be implemented by an IVUS navigation system different than IVUS navigation system 300. Further, logic flow 400 is described with reference to cardiac vasculature structure 100 and IVUS imaging system 116 of FIGS. 1A and 1B and the series of IVUS image frames 200a and longitudinal view 200b of FIGS. 2A and 2B for clarity of presentation.

Logic flow 400 can begin at block 402. At block 402 “receive a series of intravascular image (IVI) frames of a vessel of a patient” a series of IVI frames, such as, for example, IVUS image frames captured via an IVUS catheter percutaneously inserted in a vessel of a patient can be received. For example, information elements comprising indications of series of IVUS image frames 200a can be received from IVUS imaging system 116 where IVUS imaging catheter 106 is (or was) percutaneously inserted into cardiac artery 102 of cardiac vasculature structure 100. As described above, the series of IVUS image frames 200a includes several frames (e.g., IVUS image frames 202-1, 202-2, and 202-3 to 202-n) captured while the IVUS imaging catheter 106 is pulled back from the distal point 112 to proximal point 114 of the cardiac artery 102. Processor 306 can execute instructions 316 to receive information elements comprising indications of series of IVUS image frames 200a from IVUS imaging system 116, or directly from IVUS imaging catheter 106 as may be the case.

Continuing to block 404 “receive, for at least a first IVI frame of the series of IVI frames, an indication of a border of a vessel of the patient” an indication of a border of the vessel of the patient can be received for at least one IVI frame of the series of IVI frames received at block 402. For example, processor 306 can execute instructions 316 to receive indications of the borders of the vessel represented in the IVUS image frames 200a. In some embodiments, processor 306 can execute instructions 316 to automatically detect the vessel borders and store the vessel borders as detected borders 318.

Continuing to block 406 “receive, for at least a first IVI frame of the series of IVI frames, an indication of a border of a lumen of the” an indication of a border of a lumen of the vessel of the patient can be received for at least one IVI frame of the series of IVI frames received at block 402. For example, processor 306 can execute instructions 316 to receive indications of the lumen borders of the vessel represented in the IVUS image frames 200a. In some embodiments, processor 306 can execute instructions 316 to automatically detect the lumen borders and store the borders as detected borders 318. Accordingly with some embodiments, detected borders 318 can comprise indications of vessel and lumen borders.

Continuing to block 408 “generate graphical indications” graphical indications can be generated. In general, the graphical indications can include a visualization of a cross-section view of the first IVI frame, a visualization of the border of the vessel, a visualization of the border of the lumen, and a viewing icon comprising a plurality of states. For example, processor 306 can execute instructions 316 to generate graphical indications 324 comprising visualizations of the detected borders 318, manual borders 320, assessments 322, and a viewing icon having a number of states. In general, the states of the viewing icon have several states that correspond to different ones of the graphical indications 324 to be displayed, or be visible, via a GUI.

Continuing to block 410 “render a graphical user interface (GUI) from the graphical indications” a GUI can be rendered from the graphical indications generated at block 408. For example, processor 306 can execute instructions 316 to render the GUI 326 from the graphical indications 324 where the graphical indications represent various visualization as described above. In general, the GUI 326 comprises at least the visualization of the cross-section view of a frame of the IVUS image frames 200a and the viewing icon. The state of the viewing icon can dictate the other visualizations to be displayed on the GUI 326. For example, the viewing icon can include a first state (e.g., an enabled state, or the like) where the GUI 326 comprises the visualizations of the viewing icon in the first states as well as the visualizations of the border of the vessel and/or the border of the lumen. Additionally, the viewing icon can include a second state (e.g., a disabled state, or the like) where the GUI 326 comprises the visualizations of the viewing icon in the second state but does not include the visualizations of the border of the vessel or the border of the lumen.

Continuing to block 412 “send, to a display, an information element comprising indications of the GUI” the rendered GUI of block 410 can be sent to a display. For example, processor 306 can execute instructions 316 to send an information element to display 304, comprising indications of the GUI 326. The information element causes display 304 to present the GUI 326 with specific graphical indications 324, which are shown or hidden based on the current state of the viewing icon. Regardless of the state, the display 304 displays the visualization of the cross-section view and the visualization of the viewing icon in a manner consistent with the current state. Furthermore, when the viewing icon is activated, processor 306 executes instructions 316 to send a subsequent information element to display 304, comprising of the current state of the viewing icon. For example, transitioning from the first state to the second state may cause the processor 306 to execute instructions 316 to send the information element to remove the visualization of the vessel border and/or the lumen border. Furthermore, this transition from the first state to the second state may also cause the processor 306 to execute instructions 316 to send the subsequent information element to update the state of the viewing icon from the first state to the second state.

With some embodiments, processor 306 may execute instructions 316 to shade, color, or otherwise format the graphical indications (e.g., line weight, solid line, dashed line, dotted line, area shading, area color, area pattern, etc.) to indicate plaque, confidence of border detection, a detected stent, or the like. For example, the vessel border and lumen border can be shaded a different color and/or can comprise different line styles or weights. As another example, the detected borders 318 can comprise different formats (e.g., weight, style, color, etc.) than the manual borders 320.

FIG. 5A and 5B show exemplary GUIs in accordance with the various embodiments disclosed herein. For example, FIG. 5A illustrates a GUI 502a while FIG. 5B illustrates a GUI 502b. The GUIs 502a and 502b may be generated by the IVUS navigation system 300 and displayed on display 304. For example, the processor 306 can execute instructions 316 to generate the GUIs 502a and/or 502b. In general, the GUIs 502a and 502b include various visualization (e.g., based on graphical indications 324, or the like) and a viewing icon. As outlined herein, the viewing icon has a number of states that correspond to the displayed visualizations and particularly to the display of the borders (e.g., detected borders 318, manual borders 320, or the like).

For example, FIG. 5A depicts the GUI 502a and a viewing icon 504a in a first state. Further, the GUI 502a shows a cross-section visualization 506, a longitudinal vessel visualization 508, a longitudinal vessel profile visualization 510, a vessel border visualization 512, a lumen border visualization 514, and assessment visualizations 516. Additionally, the GUI 502a includes a slider 518 which can be moved along a longitudinal axis 520 to select one of the frames of the series of IVUS image frames 200a.

A user can interact with the GUIs to activate the viewing icon to change the state of the viewing icon. For example, responsive to activation (e.g., selecting, clicking, toggling, etc.) of the viewing icon 504a, processor 306 can execute instructions 316 to change the visualization displayed in the GUI 502a.

Turning to FIG. 5B, the GUI 502b is depicted. GUI 502b shows similar visualizations to the GUI 502a. However, the viewing icon 504b has a different state (e.g., a second state) and the vessel border visualization, the lumen border visualization, and the assessment visualizations are not depicted in GUI 502b. Accordingly, it can be stated that the viewing icon 504a can correspond to an enabled state (e.g., detected and/or manual borders shown) while the viewing icon 504b can correspond to a disabled state (e.g., no borders shown). Further, in some embodiments, the display of assessment visualizations 516 can correspond to the display of the borders (e.g., enabled or disabled).

As described, the viewing icon serves as a mechanism to transition between different GUI configurations. More particularly activation of the viewing icon causes the GUIs to transition between which ones of the graphical indications are displayed. Activation of the viewing icon (e.g., viewing icons 504a, 504b, or the like) may be executed in various manners, including, but not limited to clicking; touch screen interaction; mouse input; haptic feedback; physical button presses; foot pedal activation; point-based input instruments like touchpads, trackballs, joysticks, or motion sensors; tactile input components, and/or the like.

With some embodiments, the viewing icon (e.g., viewing icons 504a, 504b, or the like) can have more than two states as depicted by GUIs 502a and 502b. For example, these states may include an enabled state where all borders and assessments are displayed, a “partially enabled” state where only some of the borders are displayed (e.g., detected borders 318, manual borders 320, detected vessel border, detected lumen border, manual vessel border, manual lumen border, or some combination of these), and a disabled state where none of the borders are shown. Further, assessments 322 corresponding to the displayed borders and associated assessment visualizations 516 can be displayed. It should be noted that the specific transition sequence is not confined to the order in which the different GUI configurations are described. Rather, the transition sequence may be flexible, as opposed to fixed, to allow, among other reasons, for customization-based analysis needs.

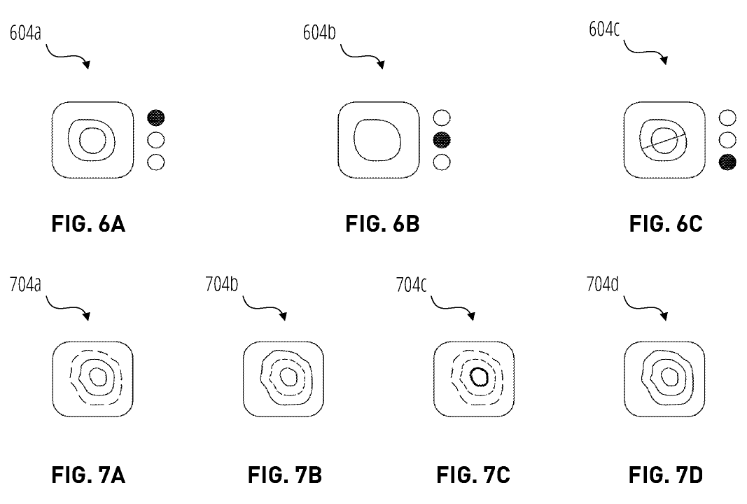

FIG. 6A, 6B and 6C illustrate an example of a viewing icon having multiple states. For example, FIG. 6A shows a viewing icon 604a that may correspond to a state where visualizations for all borders (e.g., both detected borders 318 and manual borders 320) are shown. FIG. 6B shows a viewing icon 604b that may correspond to a state where visualizations for only one type of border (e.g., detected borders 318, manual borders 320, vessel borders, lumen borders, or the like) are shown. FIG. 6C shows a viewing icon 604c that may correspond to a state where no visualizations for any borders are shown.

FIG. 7A, 7B, 7C and 7D illustrate another example of a viewing icon having multiple states. For example, FIG. 7A shows a viewing icon 704a that may correspond to a state where visualizations for detected borders 318 are shown. FIG. 7B shows a viewing icon 704b that may correspond to a state where visualizations for manual borders 320 are shown. FIG. 7C shows a viewing icon 704c that may correspond to a state where visualizations for both detected borders 318 and manual borders 320 are shown. FIG. 7D shows a viewing icon 704d that may correspond to a state where no visualizations for any borders are shown.

With some embodiments, the overall viewing functionality may maintain consistency across the various GUI configurations despite the state of the viewing icon. For example, the GUI can include various navigation menus, the cross-section visualization 506, the longitudinal vessel visualization 508, the longitudinal vessel profile visualization 510, the slider 518, the longitudinal axis 520, etc. As such, the overall layout and structure of the GUI may be maintained while toggling through the different borders associated with the frame of the series of IVUS image frames 200a depicted in the cross-section visualization 504.

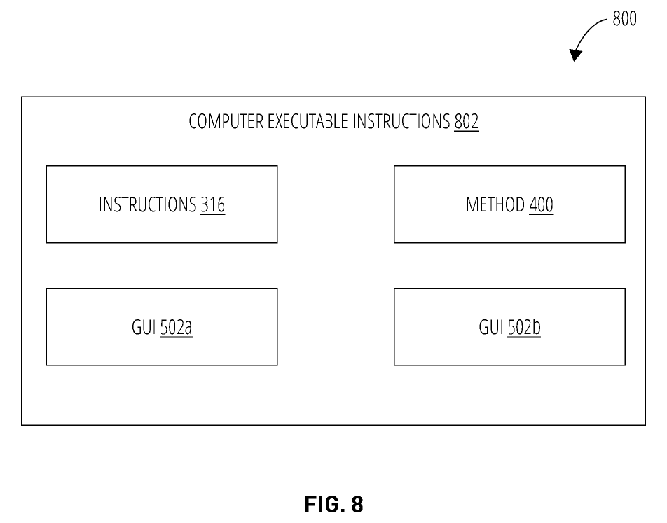

FIG. 8 illustrates computer-readable storage medium 800. Computer-readable storage medium 800 may comprise any non-transitory computer-readable storage medium or machine-readable storage medium, such as an optical, magnetic or semiconductor storage medium. In various embodiments, computer-readable storage medium 800 may comprise an article of manufacture. In some embodiments, computer-readable storage medium 800 may store computer executable instructions 802 with which circuitry (e.g., image acquisition circuitry 120, processor 306, IVUS imaging system acquisition circuitry 314, and the like) can execute. For example, computer executable instructions 802 can include instructions to implement operations described with respect to instructions 316, method 400, GUI 502a, and/or GUI 502b. Examples of computer-readable storage medium 800 or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions 802 may include any suitable type of code, such as source code, compiled code, interpreted code, executable code, static code, dynamic code, object-oriented code, visual code, and the like.

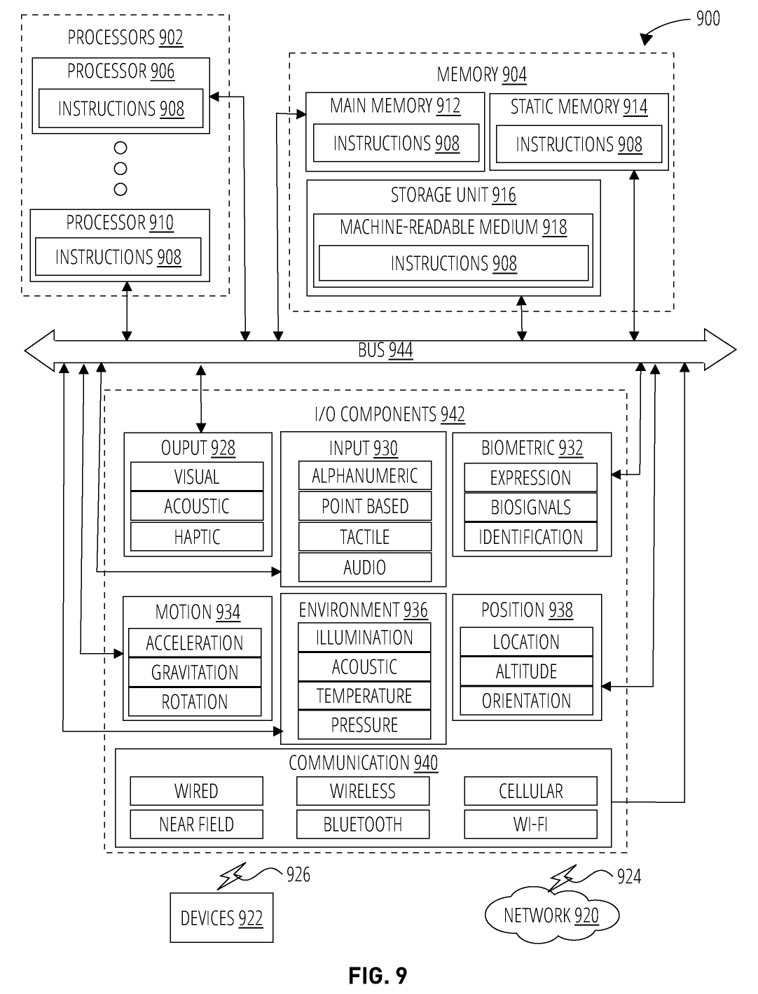

FIG. 9 illustrates a diagrammatic representation of a machine 900 in the form of a computer system within which a set of instructions may be executed for causing the machine to perform any one or more of the methodologies discussed herein. More specifically, FIG. 9 shows a diagrammatic representation of the machine 900 in the example form of a computer system, within which instructions 908 (e.g., software, a program, an application, an applet, an app, or other executable code) for causing the machine 900 to perform any one or more of the methodologies discussed herein may be executed. For example, the instructions 908 may cause the machine 900 to execute method 400, or the like. More generally, the instructions 908 may cause the machine 900 to render a GUI with a viewing icon, which may be activated to toggle the state of the viewing icon and wherein the various visualizations depicted in the GUI change based on the state of the viewing icon.

The instructions 908 transform the general, non-programmed machine 900 into a particular machine 900 programmed to carry out the described and illustrated functions in a specific manner. In alternative embodiments, the machine 900 operates as a standalone device or may be coupled (e.g., networked) to other machines. In a networked deployment, the machine 900 may operate in the capacity of a server machine or a client machine in a server-client network environment, or as a peer machine in a peer-to-peer (or distributed) network environment. The machine 900 may comprise, but not be limited to, a server computer, a client computer, a personal computer (PC), a tablet computer, a laptop computer, a netbook, a set-top box (STB), a PDA, an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 908, sequentially or otherwise, that specify actions to be taken by the machine 900. Further, while a single machine 900 is illustrated, the term “machine” shall also be taken to include several ones of machine 900 that individually or jointly execute the instructions 908 to perform any one or more of the methodologies discussed herein.

The machine 900 may include processors 902, memory 904, and I/O components 942, which may be configured to communicate with each other such as via a bus 944. In an example embodiment, the processors 902 (e.g., a Central Processing Unit (CPU), a Reduced Instruction Set Computing (RISC) processor, a Complex Instruction Set Computing (CISC) processor, a Graphics Processing Unit (GPU), a Digital Signal Processor (DSP), an ASIC, a Radio-Frequency Integrated Circuit (RFIC), another processor, or any suitable combination thereof) may include, for example, a processor 906 and a processor 910 that may execute the instructions 908. The term “processor” is intended to include multi-core processors that may comprise two or more independent processors (sometimes referred to as “cores”) that may execute instructions contemporaneously. Although FIG. 9 shows multiple processors 902, the machine 900 may include a single processor with a single core, a single processor with multiple cores (e.g., a multi-core processor), multiple processors with a single core, multiple processors with multiples cores, or any combination thereof.

The memory 904 may include a main memory 912, a static memory 914, and a storage unit 916, both accessible to the processors 902 such as via the bus 944. The main memory 904, the static memory 914, and storage unit 916 store the instructions 908 embodying any one or more of the methodologies or functions described herein. The instructions 908 may also reside, completely or partially, within the main memory 912, within the static memory 914, within machine-readable medium 918 within the storage unit 916, within at least one of the processors 902 (e.g., within the processor’s cache memory), or any suitable combination thereof, during execution thereof by the machine 900.

The I/O components 942 may include a wide variety of components to receive input, provide output, produce output, transmit information, exchange information, capture measurements, and so on. The specific I/O components 942 that are included in a particular machine will depend on the type of machine. For example, portable machines such as mobile phones will likely include a touch input device or other such input mechanisms, while a headless server machine will likely not include such a touch input device. It will be appreciated that the I/O components 942 may include many other components that are not shown in FIG. 9. The I/O components 942 are grouped according to functionality merely for simplifying the following discussion and the grouping is in no way limiting. In various example embodiments, the I/O components 942 may include output components 928 and input components 930. The output components 928 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 930 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and/or force of touches or touch gestures, or other tactile input components), audio input components (e.g., a microphone), and the like.

In further example embodiments, the I/O components 942 may include biometric components 932, motion components 934, environmental components 936, or position components 938, among a wide array of other components. For example, the biometric components 932 may include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram-based identification), and the like. The motion components 934 may include acceleration sensor components (e.g., accelerometer), gravitation sensor components, rotation sensor components (e.g., gyroscope), and so forth. The environmental components 936 may include, for example, illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 938 may include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 942 may include communication components 940 operable to couple the machine 900 to a network 920 or devices 922 via a coupling 924 and a coupling 926, respectively. For example, the communication components 940 may include a network interface component or another suitable device to interface with the network 920. In further examples, the communication components 940 may include wired communication components, wireless communication components, cellular communication components, Near Field Communication (NFC) components, Bluetooth^{®} components (e.g., Bluetooth^{®} Low Energy), Wi-Fi^{®} components, and other communication components to provide communication via other modalities. The devices 922 may be another machine or any of a wide variety of peripheral devices (e.g., a peripheral device coupled via a USB).

Moreover, the communication components 940 may detect identifiers or include components operable to detect identifiers. For example, the communication components 940 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 940, such as location via Internet Protocol (IP) geolocation, location via Wi-Fi® signal triangulation, location via detecting an NFC beacon signal that may indicate a particular location, and so forth.

The various memories (i.e., memory 904, main memory 912, static memory 914, and/or memory of the processors 902) and/or storage unit 916 may store one or more sets of instructions and data structures (e.g., software) embodying or utilized by any one or more of the methodologies or functions described herein. These instructions (e.g., the instructions 908), when executed by processors 902, cause various operations to implement the disclosed embodiments.

As used herein, the terms “machine-storage medium,” “device-storage medium,” “computer-storage medium” mean the same thing and may be used interchangeably in this disclosure. The terms refer to a single or multiple storage devices and/or media (e.g., a centralized or distributed database, and/or associated caches and servers) that store executable instructions and/or data. The terms shall accordingly be taken to include, but not be limited to, solid-state memories, and optical and magnetic media, including memory internal or external to processors. Specific examples of machine-storage media, computer-storage media and/or device-storage media include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks. The terms “machine-storage media,” “computer-storage media,” and “device-storage media” specifically exclude carrier waves, modulated data signals, and other such media, at least some of which are covered under the term “signal medium” discussed below.

In various example embodiments, one or more portions of the network 920 may be an ad hoc network, an intranet, an extranet, a VPN, a LAN, a WLAN, a WAN, a WWAN, a MAN, the Internet, a portion of the Internet, a portion of the PSTN, a plain old telephone service (POTS) network, a cellular telephone network, a wireless network, a Wi-Fi® network, another type of network, or a combination of two or more such networks. For example, the network 920 or a portion of the network 920 may include a wireless or cellular network, and the coupling 924 may be a Code Division Multiple Access (CDMA) connection, a Global System for Mobile communications (GSM) connection, or another type of cellular or wireless coupling. In this example, the coupling 924 may implement any of a variety of types of data transfer technology, such as Single Carrier Radio Transmission Technology (1xRTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard-setting organizations, other long range protocols, or other data transfer technology.

The instructions 908 may be transmitted or received over the network 920 using a transmission medium via a network interface device (e.g., a network interface component included in the communication components 940) and utilizing any one of several well-known transfer protocols (e.g., hypertext transfer protocol (HTTP)). Similarly, the instructions 908 may be transmitted or received using a transmission medium via the coupling 926 (e.g., a peer-to-peer coupling) to the devices 922. The terms “transmission medium” and “signal medium” mean the same thing and may be used interchangeably in this disclosure. The terms “transmission medium” and “signal medium” shall be taken to include any intangible medium that can store or carry the instructions 908 for execution by the machine 900 and includes digital or analog communications signals or other intangible media to facilitate communication of such software. Hence, the terms “transmission medium” and “signal medium” shall be taken to include any form of modulated data signal, carrier wave, and so forth. The term “modulated data signal” means a signal that has one or more of its characteristics set or changed in such a matter as to encode information in the signal.

With some embodiments, the viewing icon can have a default state (e.g., the enabled state, or the like). Upon initial rendering of the GUI 326, the viewing icon can be set to the default state.

Terms used herein should be accorded their ordinary meaning in the relevant arts, or the meaning indicated by their use in context, but if an express definition is provided, that meaning controls.

Herein, references to "one embodiment" or "an embodiment" do not necessarily refer to the same embodiment, although they may. Unless the context clearly requires otherwise, throughout the description and the claims, the words "comprise," "comprising," and the like are to be construed in an inclusive sense as opposed to an exclusive or exhaustive sense; that is to say, in the sense of "including, but not limited to." Words using the singular or plural number also include the plural or singular number respectively, unless expressly limited to one or multiple ones. Additionally, the words "herein," "above," "below" and words of similar import, when used in this application, refer to this application as a whole and not to any portions of this application. When the claims use the word "or" in reference to a list of two or more items, that word covers all the following interpretations of the word: any of the items in the list, all the items in the list and any combination of the items in the list, unless expressly limited to one or the other. Any terms not expressly defined herein have their conventional meaning as commonly understood by those having skill in the relevant art(s).

What is claimed is:

1. An intravascular imaging system, comprising:
a processor; and
a memory coupled to the processor, the memory comprising instructions executable by the processor, which instructions when executed cause the intravascular imaging system to:
receive, at the processor, a series of intravascular image (IVI) frames captured along a section of a cardiac artery of a patient;
receive, at the processor, for at least a first IVI frame of the series of IVI frames, an indication of a border of a vessel of the patient;
receive, at the processor, for at least the first IVI frame, an indication of a border of a lumen of the vessel;
generate a cross-section view of one of the plurality of frames;
generate a plurality of graphical indications for display, the plurality of graphical indications comprising at least:
a visualization of a cross-section view of the first IVI frame,
a visualization of the border of the vessel,
a visualization of the border of the lumen, and
a viewing icon comprising a plurality of states;
render, by the processor, a graphical user interface (GUI) from the plurality of graphical indications; and
send, by the processor to a display, an information element comprising indications of the GUI,
wherein the information element causes the display to display the GUI,
wherein, the GUI comprises at least the visualization of the cross-section view and the viewing icon,
wherein, when the viewing icon is in a first one of the plurality of states, the GUI comprises the visualization of the viewing icon in the first one of the plurality of states, the visualization of the border of the vessel and the visualization of the border of the lumen, and
wherein, when the viewing icon is in a second one of the plurality of states, the GUI comprises the visualization of the viewing icon in the second one of the plurality of states and the GUI does not comprise the visualization of the border of the vessel or the visualization of the border of the lumen.

2. The intravascular imaging system of claim 1, wherein the instructions when executed by the processor further cause the intravascular imaging system to:
receive an indication of activation of the viewing icon; and
send, to the display, a subsequent information element comprising an indication of a state of the plurality of states of the viewing icon,
wherein activation of the viewing icon changes the state of the viewing icon.

3. The intravascular imaging system of claim 1, wherein the instructions when executed by the processor further cause the intravascular imaging system to:
receive, from an input device, an indication of a manual border of the vessel; and
receive, from the input device, an indication of a manual border of the lumen,
wherein the plurality of graphical indications further comprises a visualization of the manual border of the vessel and a visualization of the manual border of the lumen,
wherein, when the viewing icon is in the second one of the plurality of states, the GUI comprises the visualization of the manual border of the vessel and the visualization of the manual border of the lumen, and
wherein, when the viewing icon is in a third one of the plurality of states, the GUI comprises the visualization of viewing icon in the third one of the plurality of states and the GUI does not comprise the visualization of the manual border of the vessel or the visualization of the manual border of the lumen.

4. The intravascular imaging system of claim 3, wherein, when the viewing icon is in the first one of the plurality of states, the GUI does not comprise the visualization of the manual border of the vessel or the visualization of the manual border of the lumen.

5. The intravascular imaging system of claim 3, wherein, when the viewing icon is in a fourth one of the plurality of states, the GUI comprises the visualization of viewing icon in the fourth one of the plurality of states and the GUI does not comprise the visualization of the border of the vessel, the visualization of the border of the lumen, the visualization of the manual border of the vessel, or the visualization of the manual border of the lumen.

6. The intravascular imaging system of claim 3, wherein, when the viewing icon is in the first one of the plurality of states, the GUI further comprises the visualization of one or more assessments.

7. The intravascular imaging system of claim 3, wherein, when the viewing icon is in the fourth one of the plurality of states, the GUI does not comprise the visualization of one or more assessments.

8. The intravascular imaging system of claim 3, wherein the processor detects the vessel border and the lumen border based on at least the first IVI frame by applying image processing techniques to at least the first IVI frame,

9. The intravascular imaging system of claim 3, wherein the processor detects the vessel border and the lumen border based on at least the first IVI frame using a machine learning model.

10. A method for an intravascular image navigation system, comprising:
receiving, at a processor, a series of intravascular image (IVI) frames captured along a section of a cardiac artery of a patient;
receiving, at the processor, for at least a first IVI frame of the series of IVI frames, an indication of a border of a vessel of the patient;
receiving, at the processor, for at least the first IVI frame, an indication of a border of a lumen of the vessel;
generating a plurality of graphical indications, the plurality of graphical indications comprising at least:
a visualization of a cross-section view of the first IVI frame,
a visualization of the border of the vessel,
a visualization of the border of the lumen, and
a viewing icon comprising a plurality of states;
rendering, by the processor, a graphical user interface (GUI) from the plurality of graphical indications; and
sending, by the processor to a display, an information element comprising indications of the GUI,
wherein the information element causes the display to display the GUI,
wherein, the GUI comprises at least the visualization of the cross-section view and the viewing icon,
wherein, when the viewing icon is in a first one of the plurality of states, the GUI comprises the visualization of the viewing icon in the first one of the plurality of states, the visualization of the border of the vessel and the visualization of the border of the lumen, and
wherein, when the viewing icon is in a second one of the plurality of states, the GUI comprises the visualization of the viewing icon in the second one of the plurality of states and the GUI does not comprise the visualization of the border of the vessel or the visualization of the border of the lumen.

11. The method of claim 10, further comprising:
receiving, at the processor, an indication of activation of the viewing icon; and
sending, by the processor to the display, a subsequent information element comprising an indication of a state of the plurality of states of the viewing icon,
wherein activation of the viewing icon changes the state of the viewing icon.

12. The method of claim 11, further comprising:
receiving, at the processor from an input device, an indication of a manual border of the vessel; and
receiving, at the processor from the input device, an indication of a manual border of the lumen,
wherein the plurality of graphical indications further comprises a visualization of the manual border of the vessel and a visualization of the manual border of the lumen,
wherein, when the viewing icon is in the second one of the plurality of states, the GUI comprises the visualization of the manual border of the vessel and the visualization of the manual border of the lumen, and
wherein, when the viewing icon is in a third one of the plurality of states, the GUI comprises the visualization of viewing icon in the third one of the plurality of states and the GUI does not comprise the visualization of the manual border of the vessel or the visualization of the manual border of the lumen.

13. The method of claim 12, wherein, when the viewing icon is in the first one of the plurality of states, the GUI does not comprise the visualization of the manual border of the vessel or the visualization of the manual border of the lumen.

14. The method of claim 12, wherein, when the viewing icon is in a fourth one of the plurality of states, the GUI comprises the visualization of viewing icon in the fourth one of the plurality of states and the GUI does not comprise the visualization of the border of the vessel, the visualization of the border of the lumen, the visualization of the manual border of the vessel, or the visualization of the manual border of the lumen.

15. The method of claim 12, further comprising receiving, at the processor, for at least the first IVI frame, an indication of one or more assessments,
wherein the plurality of graphical indications further comprises a visualization of the one or more assessments.

16. The method of claim 15, wherein, when the viewing icon is in the first one of the plurality of states, the GUI further comprises the visualization of the one or more assessments.

17. The method of claim 15, wherein, when the viewing icon is in the fourth one of the plurality of states, the GUI does not comprise the visualization of the one or more assessments.

18. The method of claim 10, further comprising detecting, by the processor, the vessel border and the lumen border based on at least the first IVI frame.

19. The method of claim 18, wherein detecting the vessel border and the lumen border based on at least the first IVI frame comprises applying image processing techniques to at least the first IVI frame.

20. The method of claim 18, wherein detecting the vessel border and the lumen border based on at least the first IVI frame comprises identifying the vessel border and the lumen border using a machine learning model.
